(19) United States
(12) Patent Application Publication
(10) Pub. No.: US 2017/0339838 A1
(43) Pub. Date: Nov. 30, 2017

(54) Air Fertilization System Directing CO2 Exhaust to a Covered Crop Row

(71) Applicant: Gerald R. Palmer, Phoenix, AZ (US)

(72) Inventor: Gerald R. Palmer, Phoenix, AZ (US)

(21) Appl. No.: 15/607,531

(22) Filed: May 28, 2017

Related U.S. Application Data
(60) Provisional application No. 62/343,008, filed on May 29, 2016.

(51) Int. Cl. A01G 7/02; C12M 1/107; C02F 3/28; C12M 1/00

(52) U.S. Cl. CPC A01G 7/02; C12M 47/18; C12M 21/04; C12M 1/107; C02F 3/2893; C02F 3/28

(57) ABSTRACT

The embodied invention is directed toward capturing air sources that contain higher concentrations of CO₂ and directing the air to a controlled crop growing environment where a crop will be able to benefit from the higher amounts of CO₂. The air is preferably filtered for dust, monitored for CO₂ levels, and a mixer is used to control the CO₂ level to a desirable amount.

### CROSS REFERENCE TO RELATED APPLICATIONS

This application claims the benefit of U.S. provisional application No. 62/343,008 filed on May 29, 2016. The entire provisional application is included by reference herein.

### STATEMENT REGARDING FEDERALLY SPONSORED RESEARCH OR DEVELOPMENT

Not applicable.

### REFERENCE TO SEQUENCE LISTING, A TABLE, OR COMPUTER PROGRAM LISTING

Not applicable.

### BACKGROUND OF THE INVENTION

### (1) Field of the Invention

This invention is directed toward air moving systems that remove CO₂ from the atmosphere by utilizing it to increase crop production.

### (2) Description of Related Art

Carbon dioxide is not considered a toxin in itself since it naturally occurs in the earth's atmosphere due to plant and animal biology. It currently occurs at levels of approximately 400 ppm (year 2015). It has been increasing in recent years due to exhaust from power plants, combustion engines, manufacturing, and other causes. The increasing CO₂ amount has become an important part of the global warming due to being a greenhouse gas. Small increases in Earth's surface temperature are projected to cause much harm to the current environmental balance.

Nitrogen (78%), oxygen (21%), argon (0.9%) and water (1%) are the other major elements of the atmosphere, and are a part of the combustion cycle. Oxygen chemically combines with carbon and hydrogen during the combustion engine or burning process, and nitrogen, argon, and water are largely inert.

The current concentration of CO2 in the atmosphere is about 400 ppm, and the increase over historical norms is the result of human activity utilizing fossil fuels as well deforestation.

In higher concentration areas such as busy roads and highways, vehicle exhaust mixes with dust and other elements to create smog (i.e. ozone). For example, Los Angeles, Mexico City, London, and China have extended periods of poor quality air, accounting for numerous lung related diseases which are projected to kill up to 5 million people per year worldwide. In addition, 9.79 billion metric tons of carbon are released into the atmosphere and creates a planet prone to global warming.

According to one study by Dr. Randall Donohue of the Commonwealth Scientific and Industrial Organization in Canberra, Australia, and his colleagues, variations of foliage on the Earth's surface were examined over a twenty-year period. They determined the amount of the carbon dioxide fertilization effect by removing all other influences, such as rainfall, and calculated that this could account for an 11 percent increase in global foliage since 1982.

Other studies agree with the general conclusion that an enhanced level of CO₂ in the ambient air will provide significant and measurable improvements in plant growth.

What is needed is a practical way to remove at least some of the carbon dioxide from the atmosphere, hopefully to benefit the planet by reducing a greenhouse gas that contributes to global warming and simultaneously improving plant growth.

### BRIEF SUMMARY OF THE INVENTION

The embodied invention is directed toward capturing air sources that contain higher concentrations of CO₂ and directing the air to a controlled crop growing environment where a crop will be able to benefit from the higher amounts of CO₂. The air is preferably filtered for dust, monitored for CO₂ levels, and a mixer is used to control the CO₂ level to a desirable amount.

### BRIEF DESCRIPTION OF THE SEVERAL VIEWS OF THE DRAWING(S)

FIG. 1 shows an overview of how the high CO₂ air is collected and distributed to crops.

FIG. 2 is a section of FIG. 1 that is taken along the lines 2-2 and shows a cross section of a plastic covered crop row.

### DETAILED DESCRIPTION OF THE INVENTION

The advantages of the present invention are due to driving improvements in photosynthesis by an increase of CO₂ levels. The higher CO₂ levels can increase plant growth and production by 15-30%. By increasing levels of CO₂ from 400 ppm up to 1000 ppm will provide proportional increases in plant growth, resulting in more sugars and carbohydrates being created by the plant. The CO₂ can be considered a supplement for plant growth and is a common practice many greenhouses.

The embodied invention captures higher concentration CO₂ air from a freeway, filters the air, and pumps it to plant rows that utilize a fabric or plastic cover. The air then exits the plant row at the opposite end of the entry side.

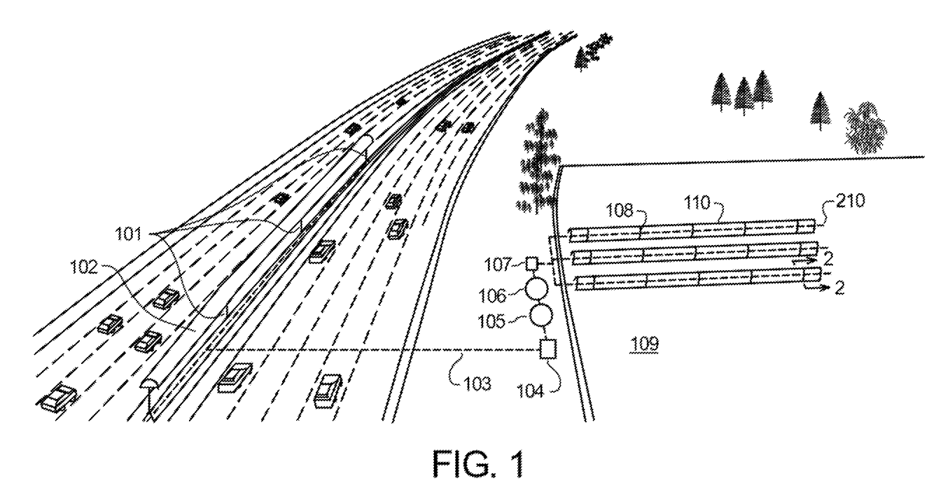

FIG. 1 shows a high CO₂ air collection pipe with slots mounted on a highway median wall 101. A long, elevated cover 102 provides high CO₂ air collection to enhance the collectable amount of high CO₂ air. The high CO₂ air will enter through slots (not shown) in the pipe or an end collection point. The high CO₂ air is then drafted through a pipe 103 under the highway to a filter 104 that removes heavier particles such as dust. The high CO₂ air is then drafted into an air moving turbine 105. The air moving turbine is preferably solar powered (solar panels not shown). The turbine provides the vacuum needed (i.e. negative 10 psi) to draw the air into the collection pipe and additionally moves the high CO₂ air through the piping. The high CO₂ air is then directed to an air volume mixer 106 which controls the amount of CO₂ in the air that will be used for growing crops. A supplement amount of CO₂ from a high pressure tank can be added if necessary to regulate the desirable amount of CO₂ in the air stream. A switch valve 107 is used to switch between the high CO₂ air and water which are both directed into the subterranean drip pipe 108. A number of covered 110 crop rows are located in a farming field 109.

The air volume mixer is a CO₂ volume mixer that provides the ideal mixture of CO₂ in the high CO₂ air stream. The CO₂ is measured and regulated by comparing to a CO₂ setpoint. Depending upon car traffic on the highway, the amount of CO₂ can vary widely. The high CO₂ air will generally be delivered to the plants during daylight hours during photosynthesis, and the water is introduced at night. The CO₂ boost delivered by the subterranean drip pipe rises above the ground and under the crop cover. This provides an enhanced CO₂ air around the plant leaves. As oxygen levels increase as a bi-product of photosynthesis, the oxygen permeates through the crop cover into the atmosphere, completing the process.

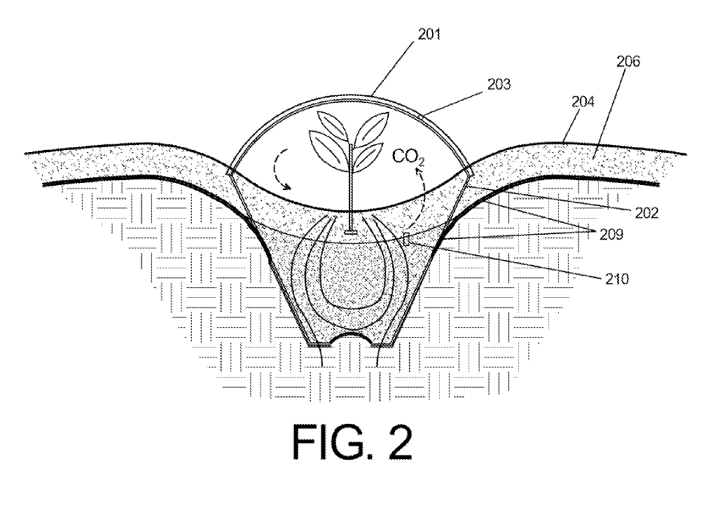

FIG. 2 is a cross section of FIG. 1 that is taken along the lines 2-2 and shows important details of a covered crop row, and is typical of the conceived invention. The need to cover the crop row with fabric or plastic is an important embodiment of the disclosed invention, as the cover provides moisture and control of the ambient air around the plants. The need for covering the crop row, or otherwise creating a controlled environment, is critical to success as moisture and CO₂ loss will negatively impact the plant growth.

A domed cover 201 protects the crop plants, and is supported by a wire or plastic frame 202 spaced at intervals along the length of the crop row. An optional small vent 203 is added to allow moisture and air to leave the covered crop row. The finish grade 204 of cover soil 206 is designed with moisture control, so as to allow rain to gather into the crop row. Various cover soil geometries are possible, based on the amount of rainfall received. An impervious plastic ground cover 209 provides soil moisture control, and provides for a growth centered plant environment. It also provides insect control. Subterranean piping or tape 210 provides for entry of water at night or high CO₂ air during day, depending upon the control of the switch valve 107 previously described.

The objective of the domed cover, control of insects, better moisture control, and enhanced CO₂ levels is improved crop growth.

While various embodiments of the present invention have been described, the invention may be modified and adapted to various operational methods to those skilled in the art. Therefore, this invention is not limited to the description and figure shown herein, and includes all such embodiments, changes, and modifications that are encompassed by the scope of the claims.

I claim:

1. A method for providing a high CO₂ air mixture for crops near a highway CO₂ source comprising:
a) providing
1) an air collection hood in a median strip of said highway,
2) collection piping located in the median strip configured to collect said high CO₂ air mixture,
3) connecting piping from said collection piping to a filter,
4) connecting piping from said filter to a turbine,
5) connecting piping from said turbine to a CO₂ mixer,
6) connecting piping from said CO₂ mixer to a switch valve, and
7) wherein said switch valve operates between water and said high CO₂ air mixture,
b) operating:
1) said air turbine to create an inlet vacuum up to 10 psi,
2) a monitor which measures a CO₂ amount in said high CO₂ air mixture,
3) said CO₂ mixer to adjust said CO₂ amount in said CO₂ air mixture according to a setpoint amount, and
4) said switch valve according to a design that will provide desirable amounts of moisture and CO₂ to a crop,
whereby said crop growth is improved according to at least one predetermined criterion.
